(19) United States
(12) Patent Application Publication
(10) Pub. No.: US 2025/0114294 A1
(43) Pub. Date: Apr. 10, 2025

(54) COMPOSITIONS SUITABLE FOR USE IN SKIN CARE

(71) Applicant: Johnson & Johnson Consumer Inc., Skillman, NJ (US)

(72) Inventors: Ramine PARSA, Lawrenceville, NJ (US); Ruchi PATEL, Dayton, NJ (US); Wen-Hwa Ting LI, Cranbury, NJ (US)

(21) Appl. No.: 18/905,643

(22) Filed: Oct. 3, 2024

Related U.S. Application Data
(60) Provisional application No. 63/588,030, filed on Oct. 5, 2023.

(51) Int. Cl. A61K 8/9789; A61K 8/67; A61Q 19/08

(52) U.S. Cl. CPC A61K 8/9789; A61K 8/671; A61Q 19/08

(57) ABSTRACT

The invention relates to composition for skin care. In particular the invention relates to skincare composition comprising a myrrh resin extract, and a retinoid. The invention also relates to a method of treating the skin using said composition, and to a method for boosting the anti-aging effects of retinoids in the skin layers.

### CROSS-REFERENCE TO RELATED APPLICATIONS

This application claims the benefit of U.S. provisional application 63/588,030 filed on Oct. 5, 2023, the complete disclosure of which is hereby incorporated herein by reference for all purposes.

### FIELD

The invention generally relates to compositions for skin care. In particular the invention relates to skincare compositions comprising a myrrh resin extract, and a retinoid. The invention also relates to a method of treating the skin using said composition.

### BACKGROUND

The human skin is subject to certain aging processes, some of which are attributable to intrinsic processes (e.g. chronoaging) and some of which are attributable to exogenous factors (e.g. photo-aging). In addition, temporary or even lasting changes to the skin can occur, such as acne, greasy or dry skin, keratoses, rosacea, light-sensitive, inflammatory, erythematous, and allergic or autoimmune-reactive reactions, such as dermatosis and photodermatosis.

The consequences of the above-mentioned aging processes can include thinning of the skin, weaker interlacing of epidermis and dermis, and a reduction in the number of cells and the supplying blood vessels. These consequences are often undesirable, and individuals suffering from these issues will look to topical treatments to address them.

Retinoids have been used for treating skin conditions caused by intrinsic aging, exogenous factors, acne or skin diseases. However, retinoids can be difficult to formulate with because many are susceptible to photodegradation and retinoids can be harsh on the skin of some individuals.

Myrrh resin is a natural extract from tree species from the genus Commiphora. Myrrh resin is known since ancient times for its application in perfume and medicine, for example as an antiseptic. Topical application of myrrh resin on the skin can cause skin irritation.

One option to avoid the irritation caused by retinoid on the skin is to propose alternative compounds which activate the same or similar biological pathways as retinoids. However, this is a complex task as such alternative compounds are difficult to identify. Another option would be to enhance, or boost, the beneficial effect of retinoids, and thus limit the amount of retinoid that is applied topically. Again, this task in complex as these compounds are difficult to identify.

There is an ongoing need and interest in identifying compounds or products that could enhance, or boost, the activation of retinoids on beneficial biological pathways, without increasing the undesirable side effects of retinoid such as skin irritation. Improving the potentiality of retinoid with a known and trusted natural ingredient would be particularly desirable.

### BRIEF SUMMARY

One aspect of the invention pertains to skincare composition comprising a myrrh resin extract and a retinoid.

This aspect may be combined with a variety of embodiments, in any combination. Thus, in some embodiments the myrrh resin extract is produced from the resin of a plant from the genus Commiphora. In one or more embodiments, the myrrh resin extract is obtained by extraction with an organic solvent. In some embodiments, the myrrh resin extract is present in an amount ranging from about 0.000001% to about 10% by total weight of the composition. In one or more embodiments, the retinoid is selected from the group consisting of retinol, retinal, retinol esters, retinoic acid, their derivatives, and combinations thereof. In one or more embodiments, the retinoid is present in an amount ranging from about 0.001% to about 10% by total weight of the composition. In one or more embodiments, the ratio of myrrh resin to retinoid is from about 100:1 to about 1:10. In some embodiments, the skincare composition further comprises an ingredient selected from the group consisting of surfactants, chelating agents, emollients, humectants, conditioners, preservatives, opacifiers, fragrances, and combinations of two or more thereof. In one or more embodiments, the skincare composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

Another aspect of the invention pertains to a skincare composition comprising about 0.01 wt. % to about 2.0 wt. % of myrrh resin extract by total weight of the composition; and about 0.01 wt. % to about 1.0 wt. % of retinol by total weight of the composition.

Another aspect of the invention pertains to a method of skin treatment comprising topically applying to the skin a composition according to any of the embodiments described above.

Another aspect of the invention pertains to a method of treating signs of skin aging, comprising topically applying to the skin in need of treatment for skin aging a skincare composition according to any of the embodiments described above.

Another aspect of the invention pertains to a method for boosting the anti-aging effects of retinoids in the skin layers of a skin in need thereof, comprising topically applying to skin a skincare composition comprising a myrrh resin extract and a retinoid.

Another aspect of the invention pertains to a method for increasing CRABP2 or AQP3 expression in skin cells, comprising topically applying to skin a skincare composition comprising a myrrh resin extract and a retinoid.

This aspect may be combined with a variety of embodiments, in any combination. Thus, in some embodiments of the method disclosed above, the retinoid is selected from the group consisting of retinol, retinal, retinol esters, retinoic acid, their derivatives, and combinations thereof. In one or more embodiments the retinol is present in an amount ranging from about 0.001% to about 10% by total weight of the composition.

In one or more embodiments the myrrh resin extract is present in an amount ranging from about 0.001% to about 10% by total weight of the composition.

In one or more embodiments the skincare composition topically applied to the skin comprises: about 0.01 wt. % to about 2.0 wt. % of myrrh resin extract by total weight of the composition; and about 0.01 wt. % to about 1.0 wt. % of retinol by total weight of the composition.

These and other features and advantages of the present invention will be readily apparent from the following detailed description of the invention.

### DETAILED DESCRIPTION

As used herein, a skin care product or skin care composition is a product that can be applied to the skin or hair of a user, providing a benefit to that user. The benefit can be, for example, hydration of the skin or hair or delivery of one or more therapeutic or benefit agents to the skin or hair, as described in more detail below.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The general convention in the scientific and technical literature is applied: the last decimal place of a numerical value indicates its degree of accuracy. Where no other error margins are given, the maximum margin is ascertained by applying the rounding-off convention to the last decimal place, for example for a measurement of 3.5%, the error margin is 3.45-3.54.

All percentages (%) are by weight unless otherwise specified herein.

One aspect of the invention pertains to a skincare composition comprising:
a myrrh resin extract, and
a retinoid.

Surprisingly this combination of myrrh resin extract and a retinoid has been found provide an increase in the CRABP2 or AQP3 expression in skin cells without the drawback such as skin irritation traditionally associated with natural extracts such as retinoids. This increase in CRABP2 or AQP3 expression is believed to provide a beneficial effect to the skin; for example, to reduce the signs of aging of the skin or increase the skin hydration.

By “extract” it is meant the substance, or the mixture of substances that is withdrawn by a chemical process from the myrrh resin. The chemical process may be for example the treatment of the myrrh resin with a solvent so as to remove a soluble substance, or a mixture of soluble substances, from the myrrh resin. The “myrrh resin extract” is thus a subset of the myrrh resin.

In one or more embodiments, the skincare composition may comprise a myrrh resin extract which is produced from the resin of a plant from the genus Commiphora.

Preferably, from the resin of plants such as Commiphora myrrha, or Commiphora molmol.

In this embodiment by “produced” it is meant that the myrrh resin is a secretion obtained from the plant bark.

The natural origin of the myrrh resin extract is an additional benefit as most consumers are looking for naturally sourced active ingredient.

In one or more embodiments, the myrrh resin extract is obtained by extraction with an organic solvent.

Myrrh resin is a complex mixture of chemical compounds, and it has been discovered that the fraction of myrrh resin isolated by an extraction with an organic solvent, such as a polar organic solvent, provides beneficial effects according to the invention.

Preferably the organic extraction of myrrh resin is performed with an alcoholic solvent such as such as methanol, ethanol, propanol, isopropanol, butanol; preferably ethanol.

The resin produced by the bark of a tree from the Commiphora genus is processed with an organic solvent, such as ethanol, to give a fraction of the said resin that is designated as “myrrh resin extract” according to the present invention.

The myrrh resin extract may also be blended with Triethyl acetylcitrate, for example the myrrh resin extract may contain from 10% to 20% Triethyl acetylcitrate, preferably from 12% to 15%.

In one or more embodiments, the myrrh resin extract is present in an amount ranging from about 0.000001% to about 10% by total weight of the composition.

Preferably the myrrh resin extract is present in an amount ranging from about 0.000001% to about 1% by total weight of the composition; or about 0.000001% to about 0.5% by total weight of the composition; or about 0.000001% to about 0.2% by total weight of the composition; or about 0.000001% to about 0.1% by total weight of the composition; or about 0.000001% to about 0.01% by total weight of the composition, or about 0.00001% to about 2% by total weight of the composition, or about 0.0001% to about 2% by total weight of the composition, or about 0.001% to about 2% by total weight of the composition, or about 0.01% to about 2% by total weight of the composition, or about 0.01% to about 1% by total weight of the composition. Preferably the myrrh resin extract is present in an amount above 0.001% by total weight of the composition. Preferably the myrrh resin extract is present in an amount above 0.005% by total weight of the composition. Preferably the myrrh resin extract is present in an amount above 0.01% by total weight of the composition. Preferably the myrrh resin extract is present in an amount below 1% by total weight of the composition. Preferably the myrrh resin extract is present in an amount below 0.5% by total weight of the composition. Preferably the myrrh resin extract is present in an amount below 0.2% by total weight of the composition. Preferably the myrrh resin extract is present in an amount below 0.1% by total weight of the composition. Preferably the myrrh resin extract is present in an amount below 0.05% by total weight of the composition. Preferably the myrrh resin extract is present in an amount below 0.02% by total weight of the composition.

In one or more embodiments, the myrrh resin extract is in a free, non-encapsulated form. Alternatively, the myrrh resin extract can be in an encapsulated form.

Preferably the myrrh resin extract is not encapsulated.

In one or more embodiments, the retinoid is selected from the group consisting of retinol, retinal, retinol esters, retinoic acid, their derivatives, and combinations thereof.

In a preferred embodiment, the retinoid is retinol.

In one or more embodiments, the retinoid is in a free, non-encapsulated form.

Alternatively, the retinoid can be in an encapsulated form.

In a preferred embodiment, the retinoid is not encapsulated.

In one or more embodiments, the retinoid is present in an amount ranging from about 0.001% to about 10% by total weight of the composition.

Preferably the retinoid is present in an amount ranging from about 0.001% to about 5%; or from about 0.001% to about 1%; from about 0.001% to about 0.5%. Preferably the retinoid is present in an amount above 0.005% by total weight of the composition. Preferably the retinoid is present in an amount above 0.01% by total weight of the composition. Preferably the retinoid is present in an amount above 0.05% by total weight of the composition. Preferably the retinoid is present in an amount above 0.1% by total weight of the composition. Preferably the retinoid is present in an amount below 5% by total weight of the composition. Preferably the retinoid is present in an amount below 1% by total weight of the composition. Preferably the retinoid is present in an amount below 0.5% by total weight of the composition.

In one or more embodiments, the ratio of myrrh resin extract to retinoid is from about 100:1 to about 1:10.

In a preferred embodiment, the ratio of myrrh resin extract to retinoid is from about 10:1 to about 1:5; preferably from about 10:1 to about 1:2; preferably from about 5:1 to about 1:1, preferably from about 2.5:1 to about 1:1; preferably about 1.25:1.

In one or more embodiments, the skincare composition according to the present

invention may further comprise an ingredient selected from the group consisting of surfactants, chelating agents, emollients, humectants, conditioners, preservatives, pearlescent or opacifiers, fragrances, and combinations of two or more thereof.

The skincare composition may include a surfactant, which may include nonionic, anionic, cationic, or amphoteric surfactants, or may include combinations of surfactants.

Examples of suitable nonionic surfactants include, but are not limited to the fatty alcohol acid or amide ethoxylates, monoglyceride ethoxylates, sorbitan ester ethoxylates alkyl polyglycosides, and mixtures thereof.

Examples of anionic surfactants include salts of alkyl phosphate, glyceryl alkyl citrate, fatty acids in the anionic form, alkyl sulfate, alkyl sarcosinate, olefin sulfonate, alkyl taurate, or alkyl isethionate.

Examples of cationic surfactants that are suitable for use in this invention include alkyl quaternaries (mono, di, or tri), benzyl quaternaries, ester quaternaries, ethoxylated quaternaries, alkyl amines, and mixtures thereof, wherein the alkyl group has from about 6 carbon atoms to about 30 carbon atoms, with about 8 to about 22 carbon atoms being preferred.

Examples of amphoteric surfactants suitable for use in the present invention include, but are not limited to, amphocarboxylates such as alkylamphoacetates (mono or di); alkyl betaines; alkylamidoalkyl betaines (other than CAPB); alkylamidoalkyl sultaines; alkylamphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof.

The surfactant, or combination of surfactants, may be present in a total amount of from about 5% to about 30% by weight of the skincare composition, or from about 10% to about 20% by weight of the skincare composition.

The skincare composition may include an emollient, where the emollient or emollient blend is useful in cleansing the skin. As used herein, “emollients” refer to materials used for cleansing the skin, hair, and eye lashes, the prevention or relief of dryness, or for the protection of the skin. Examples of emollients useful herein include, but are not limited to, linear or branched chained hydrocarbon emollients, such as decane, isohexadecane, dodecane, tridecane, tetradecane, squalene, hexadecane, or isoparaffins; hydrophobic compounds such as vegetable oils; mineral oils (e.g., petrolatum); fatty esters (e.g., isopropyl palmitate, C12-C15 alkyl benzoate) including those fatty esters of glycerol, linear or branched alkyl esters; stearic esters, such as isostearates, including isopropyl isostearate, decyl isostearate (including simple isostearates and highly mono-branched decyl isostearates, which is an ester formed from an isostearic acid having an increased proportion of mono-branched molecules compared to simple isostearic acid), and hexyl isostearate; and combinations thereof.

The skincare composition may include one emollient or may include more than one emollient. The emollient(s) may be present in a total amount of from about 3% to about 15% by weight of the skincare composition, or from about 5% to about 10% by weight of the skincare composition.

The skincare composition may be free or substantially free of antimicrobial preservatives. The skincare composition may include antioxidants, such as tocopheryl acetate. If used, antioxidants may be present in an amount of from about 0.05% to about 0.5% by weight of the skincare composition, or from about 0.1% to about 0.25% by weight of the skincare composition.

The skincare composition may additionally include one or more humectants, such as panthenol or glycerin. Humectants, if used, may be present in an amount less than about 1% by weight, or less than about 0.5% by weight, or less than about 0.25% by weight.

The skincare composition may additionally include one or more fragrances or fragrance blends, including essential oils or other fragrance ingredients. Myrrh resin extract according to the invention is not considered as a fragrance. If used, fragrances are present in a total weight of from about 0.001% to about 0.5% by weight of the composition, or from about 0.01% to about 0.2% by weight of the composition. It may be preferred that the skincare composition be free or substantially free of added colorants or dyes. It may be preferred that the skincare composition be opaque and white, avoiding having a grayish color.

Examples of suitable pearlescent or opacifying agents include, but are not limited to mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO-(JO)a-H, wherein J is an alkylene group having from about 2 to about 3 carbon atoms; and a is 2 or 3; fatty alcohols containing from about 16 to about 22 carbon atoms; fatty esters of the formula: KCOOCH₂L, wherein K and L independently contain from about 15 to about 21 carbon atoms; inorganic solids insoluble in the composition, and mixtures thereof.

In one or more embodiments, the skincare composition according to the present invention may be in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The skincare composition may be substantially free of processed oat ingredient such as oat oil.

As used herein, “essentially free” or “substantially free” of an ingredient means containing less than 0.1 weight percent, or less than 0.01 weight percent, or none of an ingredient.

Another aspect of the invention pertains to a skincare composition comprising:
a. about 0.01 wt. % to about 2.0 wt. % of myrrh resin extract by total weight of the composition; and
b. about 0.01 wt. % to about 1.0 wt. % of retinol by total weight of the composition.

The skincare composition may comprise:
a. about 0.01 wt. % to about 2.0 wt. % of myrrh resin extract by total weight of the composition; and
b. about 0.01 wt. % to about 1.0 wt. % of retinol by total weight of the composition,
wherein the myrrh resin extract is obtained by extraction with an organic solvent, from the resin of a plant from the genus Commiphora.

The skincare composition may comprise:
a. about 0.01 wt. % to about 2.0 wt. % of myrrh resin extract by total weight of the composition; and
b. about 0.01 wt. % to about 1.0 wt. % of retinol by total weight of the composition,
wherein the myrrh resin extract is obtained by extraction with an organic solvent, from the resin of a plant from the genus Commiphora, and
wherein the ratio of myrrh resin extract to retinol is from about 10:1 to about 1:5; preferably from about 10:1 to about 1:2; preferably from about 5:1 to about 1:1, preferably from about 2.5:1 to about 1:1; preferably about 1.25:1.

Another aspect of the invention pertains to a method of skin treatment comprising topically applying to the skin a skincare composition according to the present invention.

The method of skin treatment may be a cosmetic treatment of the skin.

Another aspect of the invention pertains to a method of treating sign of skin aging, comprising topically applying to the skin in need of treatment for skin aging a skincare composition according to the present invention.

The present invention is suitable for treating signs of skin aging. As used herein, “signs of skin aging” includes the presence of lines and wrinkles, loss of elasticity, uneven skin, and blotchiness. In a particularly preferred embodiment, the sign of aging is the presence of lines and wrinkles and/or loss of elasticity.

As used herein, “wrinkle” includes fine lines, fine wrinkles, or coarse wrinkles. Examples of wrinkles include, but are not limited to, fine lines around the eyes (e.g., “crow's feet”), forehead and cheek wrinkles, frown-lines, and laugh-lines around the mouth.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

In another aspect, the invention may pertain to a method of treating acne, comprising topically applying to the skin in need of treatment for acne a skincare composition according to the present invention. In a preferred embodiment the method of treating acne is a cosmetic method of acne treatment.

The invention is also suitable for treating acne. As used herein, “acne” refers to disorders resulting from the actions of hormones and other substances on the sebaceous glands and hair follicles, typically leading to clogged pores and the formation of inflammatory or non-inflammatory lesions on the skin. Specifically, it relates to blemishes, lesions, or pimples, pre-emergent pimples, blackheads, and/or whiteheads. As used herein, a “pre-emergent pimple” is an inflamed follicle that are not visually apparent on the surface of the skin with the naked eye (e.g., as a lesion).

The invention is also suitable for treating rosacea. As used herein, “rosacea” means skin with persistent erythema with or without papules, pustules, or nodules.

In another aspect, the invention may pertain to a method of smoothing skin texture, comprising topically applying to the skin in need of treatment for smoother texture a skincare composition according to the present invention. In a preferred embodiment the method of smoothing skin texture is a cosmetic method of skin texture treatment.

In another aspect, the invention may pertain to a method of brightening the skin, comprising topically applying to the skin a skincare composition according to the present invention. In a preferred embodiment the method of brightening the skin is a cosmetic method of skin treatment.

In another aspect, the invention may pertain to a method for boosting the anti-aging effects of retinoids in the skin layers of a skin in need thereof, comprising topically applying to skin a skincare composition comprising a myrrh resin extract and a retinoid.

In a preferred embodiment, the method for boosting the anti-aging effects of retinoid in the skin layers of a skin in need thereof, according to the invention, is a cosmetic treatment method.

By “boosting the anti-aging effects” it is meant that the signs of skin aging are reduced and less visible.

Another aspect of the invention pertains to a method for increasing CRABP2 or AQP3 expression in skin cells, comprising topically applying to skin a skincare composition comprising a myrrh resin extract and a retinoid.

In a preferred embodiment, the method for increasing CRABP2 or AQP3 expression in skin cells, according to the invention, is a cosmetic treatment method.

In another aspect, the invention may pertain to a skincare composition comprising a myrrh resin extract and a retinoid, for use in a cosmetic treatment method for increasing CRABP2 or AQP3 expression in skin cells.

In one or more embodiments, the method for increasing CRABP2 or AQP3 expression in skin cells further comprises a retinoid selected from the group consisting of retinol, retinal, retinol esters, retinoic acid, their derivatives, and combinations thereof.

In another aspect, the invention may pertain to a skincare composition comprising a myrrh resin extract and a retinoid, for use in a cosmetic treatment method for increasing CRABP2 or AQP3 expression in skin cells and therefore treating signs of aging, treating acne, smoothing skin texture, or brightening the skin.

In another aspect, the invention may pertain to a skincare composition comprising a myrrh resin extract and a retinoid, for use in a cosmetic treatment method for increasing CRABP2 or AQP3expression in skin cells and therefore increasing skin hydration.

Another aspect of the invention pertains to a method for increasing CRABP2 expression in skin cells, comprising topically applying to skin a skincare composition comprising a myrrh resin extract and a retinoid.

In a preferred embodiment, the method for increasing CRABP2 expression in skin cells, according to the invention, is a cosmetic treatment method.

In another aspect, the invention may pertain to a skincare composition comprising a myrrh resin extract and a retinoid, for use in a cosmetic treatment method for increasing CRABP2 expression in skin cells.

In one or more embodiments, the method for increasing CRABP2 expression in skin cells further comprises a retinoid selected from the group consisting of retinol, retinal, retinol esters, retinoic acid, their derivatives, and combinations thereof.

In another aspect, the invention may pertain to a skincare composition comprising a myrrh resin extract and a retinoid, for use in a cosmetic treatment method for increasing CRABP2 expression in skin cells and therefore treating signs of aging, treating acne, smoothing skin texture, or brightening the skin.

In another aspect, the invention may pertain to a skincare composition comprising a myrrh resin extract and a retinoid, for use in a cosmetic treatment method for increasing CRABP2 expression in skin cells and therefore increasing skin hydration.

Another aspect of the invention pertains to a method for increasing AQP3 expression in skin cells, comprising topically applying to skin a skincare composition comprising a myrrh resin extract and a retinoid.

In a preferred embodiment, the method for increasing AQP3 expression in skin cells, according to the invention, is a cosmetic treatment method.

In another aspect, the invention may pertain to a skincare composition comprising a myrrh resin extract and a retinoid, for use in a cosmetic treatment method for increasing AQP3 expression in skin cells and therefore treating signs of aging, treating acne, smoothing skin texture, or brightening the skin.

In another aspect, the invention may pertain to a skincare composition comprising a myrrh resin extract and a retinoid, for use in a cosmetic treatment method for increasing AQP3 expression in skin cells and therefore increasing skin hydration.

In one or more embodiments, the method for increasing AQP3 expression in skin cells comprises retinoid selected from the group consisting of retinol, retinal, retinol esters, retinoic acid, their derivatives, and combinations thereof.

In one or more embodiments, the method for increasing CRABP2 or AQP3 expression in skin cells comprises retinol present in an amount ranging from about 0.001% to about 10% by total weight of the composition.

Preferably the retinol is present in an amount ranging from about 0.001% to about 5%; or from about 0.001% to about 1%; from about 0.001% to about 0.5%. Preferably the retinol is present in an amount above 0.005% by total weight of the composition. Preferably the retinol is present in an amount above 0.01% by total weight of the composition. Preferably the retinol is present in an amount above 0.05% by total weight of the composition. Preferably the retinol is present in an amount above 0.1% by total weight of the composition. Preferably the retinol is present in an amount below 5% by total weight of the composition. Preferably the retinol is present in an amount below 1% by total weight of the composition. Preferably the retinol is present in an amount below 0.5% by total weight of the composition.

In one or more embodiments, the method for increasing CRABP2 or AQP3 expression in skin cells comprises myrrh resin extract present in an amount ranging from about 0.001% to about 10% by total weight of the composition.

Preferably the myrrh resin extract is present in an amount ranging from about 0.001% to about 1% by total weight of the composition; or about 0.001% to about 0.5% by total weight of the composition; or about 0.001% to about 0.2% by total weight of the composition; or about 0.001% to about 0.1% by total weight of the composition; or about 0.001% to about 0.01% by total weight of the composition, or about 0.00001% to about 2% by total weight of the composition, or about 0.0001% to about 2% by total weight of the composition, or about 0.001% to about 2% by total weight of the composition, or about 0.01% to about 2% by total weight of the composition, or about 0.01% to about 1% by total weight of the composition. Preferably the myrrh resin extract is present in an amount above 0.001% by total weight of the composition. Preferably the myrrh resin extract is present in an amount above 0.005% by total weight of the composition. Preferably the myrrh resin extract is present in an amount above 0.01% by total weight of the composition. Preferably the myrrh resin extract is present in an amount below 0.5% by total weight of the composition. Preferably the myrrh resin extract is present in an amount below 0.2% by total weight of the composition. Preferably the myrrh resin extract is present in an amount below 0.1% by total weight of the composition. Preferably the myrrh resin extract is present in an amount below 0.05% by total weight of the composition. Preferably the myrrh resin extract is present in an amount below 0.02% by total weight of the composition.

In one or more embodiments, the method for increasing CRABP2 or AQP3 expression in skin cells comprises myrrh resin extract, wherein the myrrh resin extract is obtained from Commiphora myrrha resin by extraction with an organic solvent.

In one or more embodiments, the method for increasing CRABP2 or AQP3 expression in skin cells comprises topically applying a skincare composition comprising:
a. about 0.01 wt. % to about 2.0 wt. % of myrrh resin extract by total weight of the composition; and
b. about 0.01 wt. % to about 1.0 wt. % of retinol by total weight of the composition.

Preferably the method for increasing CRABP2 or AQP3 expression in skin cells comprises topically applying a skincare composition comprising:
a. about 0.01 wt. % to about 2.0 wt. % of myrrh resin extract by total weight of the composition; and
b. about 0.01 wt. % to about 1.0 wt. % of retinol by total weight of the composition,
wherein the myrrh resin extract is obtained by extraction with an organic solvent, from the resin of a plant from the genus Commiphora.

Preferably the method for increasing CRABP2 or AQP3 expression in skin cells comprises topically applying a skincare composition comprising:
a. about 0.01 wt. % to about 2.0 wt. % of myrrh resin extract by total weight of the composition; and
b. about 0.01 wt. % to about 1.0 wt. % of retinol by total weight of the composition,
wherein the myrrh resin extract is obtained by extraction with an organic solvent, from the resin of a plant from the genus Commiphora, and
wherein the ratio of myrrh resin extract to retinol is from about 10:1 to about 1:5; preferably from about 10:1 to about 1:2; preferably from about 5:1 to about 1:1, preferably from about 2.5:1 to about 1:1; preferably about 1.25:1.

In another aspect, the invention may pertain to a method of enhancing the effect of

retinol in a skincare composition comprising the addition of a myrrh resin extract to the composition, wherein the myrrh resin extract amount for about 0.001% to about 10% by total weight of the composition.

Preferably the myrrh resin extract is present in an amount ranging from about 0.001% to about 1% by total weight of the composition; or about 0.001% to about 0.5% by total weight of the composition; or about 0.001% to about 0.2% by total weight of the composition; or about 0.001% to about 0.1% by total weight of the composition; or about 0.001% to about 0.01% by total weight of the composition, or about 0.00001% to about 2% by total weight of the composition, or about 0.0001% to about 2% by total weight of the composition, or about 0.001% to about 2% by total weight of the composition, or about 0.01% to about 2% by total weight of the composition, or about 0.01% to about 1% by total weight of the composition. Preferably the myrrh resin extract is present in an amount above 0.005% by total weight of the composition. Preferably the myrrh resin extract is present in an amount above 0.01% by total weight of the composition. Preferably the myrrh resin extract is present in an amount below 0.5% by total weight of the composition. Preferably the myrrh resin extract is present in an amount below 0.2% by total weight of the composition. Preferably the myrrh resin extract is present in an amount below 0.1% by total weight of the composition. Preferably the myrrh resin extract is present in an amount below 0.05% by total weight of the composition. Preferably the myrrh resin extract is present in an amount below 0.02% by total weight of the composition.

Preferably the retinol is present in an amount ranging from about 0.001% to about 5%; or from about 0.001% to about 1%; from about 0.001% to about 0.5%. Preferably the retinol is present in an amount above 0.005% by total weight of the composition. Preferably the retinol is present in an amount above 0.01% by total weight of the composition. Preferably the retinol is present in an amount above 0.05% by total weight of the composition. Preferably the retinol is present in an amount above 0.1% by total weight of the composition. Preferably the retinol is present in an amount below 5% by total weight of the composition. Preferably the retinol is present in an amount below 1% by total weight of the composition. Preferably the retinol is present in an amount below 0.5% by total weight of the composition.

Preferably the ratio of myrrh resin extract to retinol is from about 10:1 to about 1:5; preferably from about 10:1 to about 1:2; preferably from about 5:1 to about 1:1, preferably from about 2.5:1 to about 1:1; preferably about 1.25:1.

The present invention may be better understood through the following examples, which are exemplary in nature and not intended to be limiting to any specific combination of elements.

### EXAMPLES

### Methods

EpiDerm Full Thickness Skin 3D Equivalents (EFT-400) were purchased from MatTek Corporation (Ashland, MA 01721; lot number 34272, kit N & kit P). Upon receipt the skin equivalents were transferred in new 6-well plates (MatTek Corporation, Ashland, MA 01721) with 2.5 mL of EFT-400 medium (MatTek Corportion, Ashland, MA) and equilibrated overnight.

The following day, skin equivalents were treated topically with 4 μL of the following treatments: Vehicle, 0.04% Retinol, 0.04% Retinol +0.05% Myrrh resin extract, or 0.04% Retinol +0.1% Myrrh resin extract.

Propylene glycol/ethanol (PgETOH) (3:7) (w/w) was used as a vehicle. Retinol (BASF 50C) was diluted in PgETOH to a stock concentration of 0.4%. 0.4% Retinol was further diluted to make 0.08% Retinol. Since Retinol is 50% active, the percent active Retinol in the final treatment solution was 0.04% Retinol. Myrrh resin extract (Firmenich) was weighed out to 16.1 mg and diluted in 1.6 mL of PgETOH for a 1% Myrrh resin extract stock solution.

To make the 0.04% Retinol+0.1% Myrrh resin extract treatment solution combine 100 μL of the 0.4% retinol stock solution, 50 μL of the 1% Myrrh resin extract stock solution, and 350 μL of PgETOH. To make the 0.04% Retinol+0.05% Myrrh resin extract treatment solution combine 100 μL of the 0.4% retinol stock solution, 25 μL of the 1% Myrrh resin extract stock solution, and 375 μL of PgETOH.

After 24 hours of treatment the EFT-400 skin equivalents were cut out of their inserts and lysed in 600 μL lysis buffer, consisting of 100 parts RLT buffer (RNA purification kit, sold under the tradename RNEASY Mini kit, Qiagen, Valencia, CA), to one part 2-mercaptoethanol inside a reinforced tube with screw cap and o-ring closure, and beads in the tube for tissue grinding (sold under the tradename PRECELLYS CKMix50, Bertin Corp, Rockville, MD). The tubes were shaken 30 seconds at 7200 RPM for 4 cycles with a 40 second pause in between cycles. RNA was extracted from the solutions using the RNEASY Mini Kit (Qiagen, Valencia, CA) and according to manufacturer's instructions and RNA was eluted in 30 μL RNase-free water. Reverse transcription (RT) was performed using the Invitrogen SuperScript IV ViLo kit (ThermoFisher Scientific, Bridgewater, NJ).

Gene expression was performed using the gene expression assays sold under the tradename TAQMAN for cellular retinoic acid binding protein 2 (CRABP2), aquaporin-3 (AQP3), interleukin-8 (IL8), and polymerase (RNA) II polypeptide A (POLR2A), and fast advanced master mix sold under the tradename TAQMAN (ThermoFisher Scientific, Bridgewater, NJ). qPCR analysis was performed using the TAQMAN fast advanced master mix and run on a real time PCR system sold under the tradename QUANTSTUDIO 7 Flex System (ThermoFisher Scientific, Bridgewater, NJ).

The expression of these genes was normalized against the expression of the human POLR2A “housekeeping” gene. This “housekeeping” gene is a widely used control for normalizing specific gene expression levels because of their invariant expression across tissues, cells, and experimental treatments. The method for normalization involves measuring the expression of an internal reference or “housekeeping” gene, which takes into account the potential error of RNA/cDNA loading, and variation of reverse transcription efficiency.

The fold changes were calculated in comparison to the vehicle control and two-tailed two-sample Student t-tests (Microsoft Office Excel 2007; Microsoft, Redmond, WA, USA) were performed.

Results are shown as Average Fold Change over vehicle in Tables 1-3.

“Untreated” means that the gene expression assay was performed with the vehicle: Propylene glycol/ethanol (PgETOH), without Retinol or Myrrh resin extract.

### Results

TABLE 1
CRABP2 Average Fold Change over Untreated

| | | Average | |
|---|---|---|---|
| | | fold change | |
| | | over | standard |
| Crabp2 | Treatment | untreated | deviation |
| Sample 1 (comp) | Untreated (PgETOH) | 1.00 | 0.39 |
| Sample 2 (comp) | 0.04% Retinol | 2.22 | 0.60 |
| Sample 3 | 0.04% Retinol + 0.05% Myrrh resin extract | 3.67 | 0.39 |
| Sample 4 | 0.04% Retinol + 0.1% Myrrh resin extract | 2.85 | 0.47 |

TABLE 2
AQP3 Average Fold Change over Untreated

| | | Average | |
|---|---|---|---|
| | | fold change | |
| | | over | standard |
| AQP3 | | untreated | deviation |
| Sample 1 | PgETOH | 1.00 | 0.00 |
| (comp) | | | |
| Sample 2 | 0.04% Retinol | 1.82 | 1.10 |
| (comp) | | | |
| Sample 3 | 0.04% Retinol + 0.05% Myrrh resin extract | 6.53 | 1.47 |
| Sample 4 | 0.04% Retinol + 0.1% Myrrh resin extract | 7.57 | 0.03 |

TABLE 3
IL8 Average Fold Change over Untreated

| | | Average | |
|---|---|---|---|
| | | fold change | |
| | | over | standard |
| IL8 | | untreated | deviation |
| Sample 1 | PgETOH | 1.00 | 0.39 |
| (comp) | | | |
| Sample 2 | 0.04% Retinol | 1.22 | 0.44 |
| (comp) | | | |
| Sample 3 | 0.04% Retinol + 0.05% Myrrh resin extract | 1.33 | 0.57 |
| Sample 4 | 0.04% Retinol + 0.1% Myrrh resin extract | 1.11 | 0.11 |

### Conclusions

CRABP2 is well-established in literature as a highly sensitive marker of retinoid bioactivity and potency (Elder J T, Cromie M A, Griffiths C E M, Chambon P, Voorhees J J. Stimulus-selective induction of CRABP-II mRNA: A marker for retinoic acid action in human skin. J Invest Dermatol. 1993; 100(4), and has been associated with conferring skin anti-aging benefits (Bielli A, Scioli M G, D'Amico F, Tarquini C, Agostinelli S, Costanza G, Doldo E, Campione E, Passeri D, Coniglione F, Orlandi A. Cellular retinoic acid binding protein-II expression and its potential role in skin aging. Aging (Albany NY). 2019 Mar. 18; 11(6):1619-1632).

Surprisingly, the combination of 0.04% Retinol+0.05% Myrrh resin extract shows a significantly higher CRABP2 induction than Retinol alone.

This data shows the combination of Retinol and Myrrh resin extract results in an increased retinol-like property, and hence antiaging benefits. Each biomarker being associated with anti-aging benefits, such an added benefit of the combination is very advantageous.

In parallel with the increased CRABP2 induction, no increase in terms of IL8 is observed in most combinations. Retinoids are known to result in irritation and stinging upon application, which in vitro would be seen as IL8 induction. Our results indicated that even though the combination results in increased retinol-like activity, it does so without the common inflammation side-effect.

Aquaporin 3 (AQP3) is a passive transporter of water, which is associated with skin hydration and is important for maintaining water homeostasis in the skin. AQP3 is indirectly induced by retinol. The results above demonstrate that the combinations of Retinol with Myrrh resin extract show a significantly increased induction of AQP3 when compared to Retinol alone. Our results indicate the combination shows an increased benefit in skin hydration.

It has been observed that an active dose of a compound, for example myrrh resin extract or retinol, providing an in-vitro benefit may generally requires a 10-fold increase in-vivo to demonstrate similar benefits.

1. A skin care composition comprising:
a. a myrrh resin extract, and
b. a retinoid.

2. The skincare composition of claim 1, wherein the myrrh resin extract is produced from the resin of a plant from the genus Commiphora.

3. The skincare composition of claim 2, wherein the myrrh resin extract is obtained by extraction with an organic solvent.

4. The skincare composition according to claim 1, wherein the myrrh resin extract is present in an amount ranging from about 0.000001% to about 10% by total weight of the composition.

5. The skincare composition according to claim 1, wherein the retinoid is selected from the group consisting of retinol, retinal, retinol esters, retinoic acid, their derivatives, and combinations thereof.

6. The skincare composition according to claim 1, wherein the retinoid is present in an amount ranging from about 0.001% to about 10% by total weight of the composition.

7. The skincare composition according to claim 1, wherein the ratio of myrrh resin to retinoid is from about 100:1 to about 1:10.

8. The skincare composition according to claim 1, further comprising an ingredient selected from the group consisting of surfactants, chelating agents, emollients, humectants, conditioners, preservatives, opacifiers, fragrances, and combinations of two or more thereof.

9. The skincare composition according to claim 1, wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

10. The skincare composition of claim 1, comprising:
a. about 0.01 wt. % to about 2.0 wt. % of myrrh resin extract by total weight of the composition; and
b. about 0.01 wt. % to about 1.0 wt. % of retinol by total weight of the composition.

11. A method of skin treatment comprising topically applying to the skin a composition according to claim 1.

12. A method of treating sign of skin aging, comprising topically applying to the skin in need of treatment for skin aging a skincare composition according to claim 1.

13. A method for boosting the anti-aging effects of retinoids in the skin layers of a skin in need thereof, comprising topically applying to skin a skincare composition according to claim 1.

14. A method for increasing CRABP2 or AQP3 expression in skin cells, comprising topically applying to skin a skincare composition according to claim 1.

15. The method according to claim 14, wherein the retinoid is selected from the group consisting of retinol, retinal, retinol esters, retinoic acid, their derivatives, and combinations thereof.

16. The method according to claim 14, wherein the retinol is present in an amount ranging from about 0.001% to about 10% by total weight of the composition.

17. The method according to claim 14, wherein the myrrh resin extract is present in an amount ranging from about 0.001% to about 10% by total weight of the composition.

18. The method according to claim 14, wherein the skincare composition topically applied to the skin comprises:
a. about 0.01 wt. % to about 2.0 wt. % of myrrh resin extract by total weight of the composition; and
b. about 0.01 wt. % to about 1.0 wt. % of retinol by total weight of the composition.
